(19) United States
(12) Patent Application Publication
(10) Pub. No.: US 2006/0264651 A1
(43) Pub. Date: Nov. 23, 2006

(54) Method for hydrocyanating an olefinically unsaturated compound

(75) Inventors: Michael Bartsch, Neustadt (DE); Robert Baumann, Mannheim (DE); Gerd Haderlein, Grunstadt (DE); Miguel Angel Flores, Aranjuez (Madrid) (ES); Tim Jungkamp, Kapellen (BE); Hermann Luyken, Ludwigshafen (DE); Jens Scheidel, Hirschberg (DE); Wolfgang Siegel, Limburgerhof (DE); Peter Babier, Vierheim (DE)

Correspondence Address: CONNOLLY BOVE LODGE & HUTZ LLP, P.O. BOX 2207, WILMINGTON, DE, 19899-2207, US

(73) Assignee: Basf Aktiengesellschaft Patents, Trademarks and Licenses, Ludwigshafen (DE)

(21) Appl. No.: 10/548,536

(22) PCT Filed: Feb. 24, 2004
(86) PCT No.: PCT/EP04/01802

§ 371 Date: Sep. 12, 2005

(30) Foreign Application Priority Data
Mar. 12, 2003 (DE) 103 11 122.0

(51) Int. Cl. C07C 253/10

(52) U.S. Cl. 558/338

(57) ABSTRACT

A process for hydrocyanating an olefinically unsaturated nitrile in the presence of an Ni(0)-containing catalyst, which comprises carrying out the reaction in the presence of a hydrocarbon which leads under certain pressure, concentration and temperature conditions to the formation of at least two liquid phases of the overall system, of which one phase has a higher proportion of the Ni(0)-containing catalyst, based on the total weight of this phase, than the other phase or other phases.

The present invention relates to a process for hydrocyanating an olefinically unsaturated nitrile in the presence of an Ni(0)-containing catalyst, which comprises carrying out the reaction in the presence of a hydrocarbon which leads under certain pressure, concentration and temperature conditions to the formation of at least two liquid phases of the overall system, of which one phase has a higher proportion of the Ni(0)-containing catalyst, based on the total weight of this phase, than the other phase or other phases.

Processes for hydrocyanating an olefinically unsaturated nitrile in the presence of an Ni(0)-containing catalyst are known.

For instance, U.S. Pat. No. 3,773,809 describes the hydrocyanation of 3-pentenenitrile or 4-pentenenitrile in the presence of a catalyst system composed of Ni(0) and one of these complexing ligand systems comprising firstly monophosphines or monophosphites and secondly a nitrile, and also further compounds as catalyst promotors.

The resulting product mixture is admixed with a hydrocarbon in an extractor under defined conditions to form a multiphasic system. One phase of this multiphasic system comprises the hydrocarbon and the predominant portion of the organophosphorus compounds and the Ni(0) complexes mentioned, while organic mononitrile, organic dinitrile, decomposed Ni catalyst, decomposed organophosphorus compound and catalyst promoter are substantially present in another phase.

The hydrocarbon phase is removed.

Organic mononitrile, organic dinitrile and catalyst promoter are removed from the decomposed nickel catalyst and the decomposed organophosphorus compound in the other phase.

With regard to the retention or utilization of the hydrocarbon phase, U.S. Pat. No. 3,773,809 merely contains the information in example 3 that the hydrocarbon was removed to obtain a concentrate.

A disadvantage of such a distillative removal of the hydrocarbon is that the content of extractable product in the extractant is only low. According to example 3, only 4.61 g of extractable product are present in 4638 g of cyclohexane. The removal mentioned is therefore associated with high energy and technical demands.

In addition, this distillative removal has the problem that on the one hand, to prevent thermal decomposition of the catalytically active compounds present in the hydrocarbon phase, a very low distillation temperature is desirable, as attained, for example, by reducing the pressure; on the other hand, it is desirable in industrial distillations to use river water for countercooling, i.e. for condensing the distillate. This in turn sets limits on the reduction of the distillation pressure.

It is an object of the present invention to provide a process which enables the removal of the Ni(0)-containing catalysts used in the hydrocyanation of an olefinically unsaturated nitrile from the product and unconverted reactant, preferably with the possibility of reusing the catalyst mentioned, in particular in the hydrocyanation mentioned, in a technically simple and economic manner.

We have found that this object is achieved by the process defined at the outset.

According to the invention, an olefinically unsaturated nitrile is hydrocyanated in the presence of an Ni(0)-containing catalyst.

The preparation of Ni(0)-containing catalyst systems is known per se and, for the purposes of the present invention, can be effected by processes known per se.

In a preferred embodiment, the Ni(0)-containing catalyst may additionally contain a compound which is suitable as a ligand for Ni(0) and contains at least one trivalent phosphorus atom, or a mixture of such compounds.

In a preferred embodiment, the compound used as a ligand may be one of the formula
P(X¹R¹)(X²R²)(X³R³) (I).

In the context of the present invention, this compound is a single compound or a mixture of different compounds of the aforementioned formula.

X¹, X², X³ may each independently be oxygen or a single bond.

When all of the X¹, X² and X³ groups are single bonds, compound (I) is a phosphine of the formula P(R¹ R² R³) with the definitions of R¹, R² and R³ specified in this description.

When two of the X¹, X² and X³ groups are single bonds and one is oxygen, compound (I) is a phosphinite of the formula P(OR¹)(R²)(R³) or P(R¹)(OR²)(R³) or P(R¹)(R²)(OR³) with the definitions of R¹, R² and R³ specified in this description.

When one of the X¹, X² and X³ groups is a single bond and two are oxygen, compound (I) is a phosphonite of the formula P(OR¹)(OR²)(R³) or P(R¹)(OR²)(OR³) or P(OR¹)(R²)(OR³) with the definitions of R¹, R² and R³ specified in this description.

In a preferred embodiment, all X¹, X² and X³ groups should be oxygen, so that compound (I) is advantageously a phosphite of the formula P(OR¹)(OR²)(OR³) with the definitions of R¹, R² and R³ specified in this description.

According to the invention, R¹, R², R³ are each independently identical or different organic radicals.

R¹, R² and R³ are each independently alkyl radicals, advantageously having from 1 to 10 carbon atoms, such as methyl, ethyl, n-propyl, i-propyl, n-butyl, i-butyl, s-butyl, t-butyl, aryl groups such as phenyl, o-tolyl, m-tolyl, p-tolyl, 1-naphthyl, 2-naphthyl, or hydrocarbyl, advantageously having from 1 to 20 carbon atoms, such as 1,1′-biphenol, 1,1′-binaphthol.

The R¹, R² and R³ groups may be bonded together directly, i.e. not solely via the central phosphorus atom. Preference is given to the R¹, R² and R³ groups not being bonded together directly.

In a preferred embodiment, R¹, R² and R³ are radicals selected from the group consisting of phenyl, o-tolyl, m-tolyl and p-tolyl.

In a particularly preferred embodiment, a maximum of two of the R¹, R² and R³ groups should be phenyl groups.

In another preferred embodiment, a maximum of two of the R¹, R² and R³ groups should be o-tolyl groups.

Particularly preferred compounds which may be used are those of the formula
(o-tolyl-O-)_{w}(m-tolyl-O-)ₓ(p-tolyl-O-)_{y}(phenyl-O—)_{z}P

where w, x, y, z are each a natural number
where w+x+y+z=3and
   w, z are each less than or equal to 2,
      such as (p-tolyl-O-)(phenyl)₂P, (m-tolyl-O-)(phenyl)₂P, (o-tolyl-O-)(phenyl)₂P, (p-tolyl-O-)₂(phenyl)P, (m-tolyl-O-)₂(phenyl)P, (o-tolyl-O-)₂(phenyl)P, (m-tolyl-O-)(p-tolyl-O-)(phenyl)P, (o-tolyl-O-)(p-tolyl-O-)(phenyl)P, (o-tolyl-O-)(m-tolyl-O-)(phenyl)P, (p-tolyl-O—)₃P, (m-tolyl-O-)(p-tolyl-O—)₂P, (o-tolyl-O-)(p-tolyl-O—)₂P, (m-tolyl-O-)₂(p-tolyl-O—)P, (o-tolyl-O-)₂(p-tolyl-O—)P, (o-tolyl-O-)(m-tolyl-O-)(p-tolyl-O—)P, (m-tolyl-O—)₃P, (o-tolyl-O-)(m-tolyl-O—)₂P (o-tolyl-O-)₂(m-tolyl-O—)P or mixtures of such compounds.

For example, mixtures comprising (M-tolyl-O—)₃P, (M-tolyl-O-)₂(p-tolyl-O—)P, (m-tolyl-O-)(p-tolyl-O—)₂P and (p-tolyl-O—)₃P may be obtained by reacting a mixture comprising m-cresol and p-cresol, in particular in a molar ratio of 2:1, as obtained in the distillative workup of crude oil, with a phosphorus trihalide, such as phosphorus trichloride.

Such compounds and their preparation are known per se.

In a further preferred embodiment, the compound suitable as a ligand for Ni(0) which is used may be one of the formula 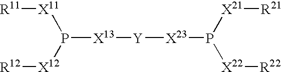
where
X¹¹, X¹², X¹³ X²¹, X²², X²³ are each independently oxygen or a single bond
R¹¹, R¹² are each independently identical or different, individual or bridged organic radicals
R²¹, R²² are each independently identicai or different, individual or bridged organic radicals,
Y is a bridging group.

In the context of the present invention, such a compound is a single compound or a mixture of different compounds of the aforementioned formula.

In a preferred embodiment, X¹¹, X¹², X¹³, X²¹, X²², X²³ may each be oxygen. In such a case, the bridging group Y is bonded to phosphite groups.

In another preferred embodiment, X¹¹ and X¹² may each be oxygen and X¹³ a single bond, or X¹¹ and X¹³ oxygen and X¹² a single bond, so that the phosphorus atom surrounded by X¹¹, X¹² and X¹³ is the central atom of a phosphonite. In such a case, X²¹, X²² and X²³ may be oxygen, or X²¹ and X²² may each be oxygen and X²³ a single bond, or X²¹ and X²³ may each be oxygen and X²² a single bond, or X²³ may be oxygen and X²¹ and X²² each a single bond, or X²¹ may be oxygen and X²² and X²³ each a single bond, or X²¹, X²² and X²³ may each be a single bond, so that the phosphorus atom surrounded by X²¹, X²² and X²³ may be the central atom of a phosphite, phosphonite, phosphinite or phosphine, preferably a phosphonite.

In another preferred embodiment, X¹³ may be oxygen and X¹¹ and X¹² each a single bond, or X¹¹ may be oxygen and X¹² and X¹³ each a single bond, so that the phosphorus atom surrounded by X¹¹, X¹² and X¹³ is the central atom of a phosphinite. In such a case, X²¹, X²² and X²³ may each be oxygen, or X²³ may be oxygen and X²¹ and X²² a single bond, or X²¹ may be oxygen and X²² and X²³ each a single bond, or X²¹, X²² and X²³ may each be a single bond, so that the phosphorus atom surrounded by X²¹, X²² and X²³ may be the central atom of a phosphite, phosphinite or phosphine, preferably a phosphinite.

In another preferred embodiment, X¹¹, X¹² and X¹³ may each be a single bond, so that the phosphorus atom surrounded by X¹¹, X¹² and X¹³ is the central atom of a phosphine. In such a case, X²¹, X²² and X²³ may each be oxygen, or X²¹, X²² and X²³ may each be a single bond, so that the phosphorus atom surrounded by X²¹, X²² and X²³ may be the central atom of a phosphite or phosphine, preferably a phosphine.

The bridging group Y is advantageously an aryl group which is substituted, for example by C₁-C₄-alkyl, halogen, such as fluorine, chlorine, bromine, halogenated alkyl, such as trifluoromethyl, aryl, such as phenyl, or is unsubstituted, preferably a group having from 6 to 20 carbon atoms in the aromatic system, in particular pyrocatechol, bis(phenol) or bis(naphthol).

The R¹¹ and R¹² radicals may each independently be the same or different organic radicals. Advantageous R¹¹ and R¹² radicals are aryl radicals, preferably those having from 6 to 10 carbon atoms, which may be unsubstituted or mono- or polysubstituted, in particular by C1-C4-alkyl, halogen, such as fluorine, chlorine, bromine, halogenated alkyl, such as trifluoromethyl, aryl, such as phenyl, or unsubstituted aryl groups.

The R²¹ and R²² radicals may each independently be the same or different organic radicals. Advantageous R²¹ and R²² radicals are aryl radicals, preferably those having from 6 to 10 carbon atoms, which may be unsubstituted or mono- or polysubstituted, in particular by C1-C4-alkyl, halogen, such as fluorine, chlorine, bromine, halogenated alkyl, such as trifluoromethyl, aryl, such as phenyl, or unsubstituted aryl groups.

The R¹¹ and R¹² radicals may each be separate or bridged.

The R²¹ and R²² radicals may each be separate or bridged.

The R¹¹, R¹², R²¹ and R²² radicals may each be separate, two may be bridged and two separate, or all four may be bridged, in the manner described.

In a particularly preferred embodiment, useful compounds are those of the formula I, II, III, IV and V specified in U.S. Pat. No. 5,723,641.

In a particularly preferred embodiment, useful compounds are those of the formula I, II, III, IV, V, VI and VII specified in U.S. Pat. No. 5,512,696, in particular the compounds used there in examples 1 to 31.

In a particularly preferred embodiment, useful compounds are those of the formula I, II, III, IV, V, VI, VII, VIII, IX, X, XI, XII, XIII, XIV and XV specified in U.S. Pat. No. 5,821,378, in particular the compounds used there in examples 1 to 73.

In a particularly preferred embodiment, useful compounds are those of the formula I, II, III, IV, V and VI specified in U.S. Pat. No. 5,512,695, in particular the compounds used there in examples 1 to 6.

In a particularly preferred embodiment, useful compounds are those of the formula I, II, III, IV, V, VI, VII, VIII, IX, X, XI, XII, XIII and XIV specified in U.S. Pat. No. 5,981,772, in particular the compounds used there in examples 1 to 66.

In a particularly preferred embodiment, useful compounds are those specified in U.S. Pat. No. 6,127,567 and the compounds used there in examples 1 to 29.

In a particularly preferred embodiment, useful compounds are those of the formula I, II, III, IV, V, VI, VII, VIII, IX and X specified in U.S. Pat. No. 6,020,516, in particular the compounds used there in examples 1 to 33.

In a particularly preferred embodiment, useful compounds are those specified in U.S. Pat. No. 5,959,135, and the compounds used there in examples 1 to 13.

In a particularly preferred embodiment, useful compounds are those of the formula I, II and III specified in U.S. Pat. No. 5,847,191.

In a particularly preferred embodiment, useful compounds are those specified in U.S. Pat. No. 5,523,453, in particular the compounds illustrated there in formula 1, 2, 3, 4, 5, 6, 7, 8, 9, 10, 11, 12, 13, 14, 15, 16, 17, 18, 19, 20 and 21.

In a particularly preferred embodiment, useful compounds are those specified in WO 01/14392, preferably the compounds illustrated there in formula V, VI, VII, VIII, IX, X, XI, XII, XIII, XIV, XV, XVI, XVII, XXI, XXII, XXIII.

In a particularly preferred embodiment, useful compounds are those specified in WO 98/27054.

In a particularly preferred embodiment, useful compounds are those specified in WO 99/13983.

In a particularly preferred embodiment, useful compounds are those specified in WO 99/64155.

In a particularly preferred embodiment, useful compounds are those specified in the German laid-open specification DE 10038037.

In a particularly preferred embodiment, useful compounds are those specified in the German laid-open specification DE 10046025.

Such compounds and their preparation are known per se.

In a further preferred embodiment, a mixture of one or more of the aforementioned compounds which are suitable as a ligand for Ni(0) and contain one phosphorus atom, and one or more compounds which are suitable as a ligand for Ni(0) and contain two phosphorus atoms may be used.

In a particularly preferred embodiment, useful systems are those which are specified in the international patent application PCT/EP02/07888 and comprise Ni(0) and such mixtures.

In a preferred embodiment, the hydrocyanation can be carried out in the presence of a Lewis acid.

In the context of the present invention, a Lewis acid is either a single Lewis acid or else a mixture of a plurality of, for example two, three or four, Lewis acids.

Processes for hydrocyanating an olefinically unsaturated nitrile, in particular the preparation of adiponitrile by hydrocyanating an olefinically unsaturated compound such as 2-cis-pentenenitrile, 2-trans-pentenenitrile, 3-cis-pentenenitrile, 3-trans-pentenenitrile, 4-pentenenitrile, E-2-methyl-2-butenenitrile, Z-2-methyl-2-butenenitrile, 2-methyl-3-butenenitrile or mixtures thereof, in the presence of a catalyst system comprising a Lewis acid and a complex containing a phosphorus compound suitable as a ligand, such as a monodentate, preferably multidentate, in particular bidentate compound which coordinates with a central atom via a phosphorus atom and which may be present as a phosphine, phosphite, phosphonite or phosphinite or a mixture thereof, and a central atom, preferably nickel, cobalt or palladium, in particular nickel, more preferably in the form of nickel(0) are known, for example from U.S. Pat. No. 4,705,881, U.S. Pat. No. 6,127,567, U.S. Pat. No. 6,171,996 B1 and U.S. Pat. No. 6,380,421 B1.

Useful Lewis acids are inorganic or organic metal compounds in which the cation is selected from the group consisting of scandium, titanium, vanadium, chromium, manganese, iron, cobalt, copper, zinc, boron, aluminum, yttrium, zirconium, niobium, molybdenum, cadmium, rhenium and tin. Examples include ZnBr₂, ZnI₂, ZnCl₂, ZnSO₄, CuCl₂, CuCl, Cu(O₃SCF₃)₂, CoCl₂, CoI₂, FeI₂, FeCl₃, FeCl₂, FeCl₂(THF)₂, TiCl₄(THF)₂, TiCl₄, TiCl₃, ClTi(O-i-propyl)₃, MnCl₂, ScCl₃, AlCl₃, (C₈H₁₇)AlCl₂, (C₈H₁₇)₂AlCl, (i-C₄H₉)₂AlCl, (C₆H₅)₂AlCl, (C₆H₅)AlCl₂, ReCl₅, ZrCl₄, NbCl₅, VCl₃, CrCl₂, MoCl₅, YCl₃, CdCl₂, LaCl₃, Er(O₃SCF₃)₃, Yb(O₂CCF₃)₃, SmCl₃, B(C₆H₅)₃, TaCl₅, as described, for example, in U.S. Pat. No. 6,127,567, U.S. Pat. No. 6,171,996 and U.S. Pat. No. 6,380,421. Also useful are metal salts such as ZnCl₂, Col₂ and SnCl₂, and organometallic compounds such as RAlCl₂, R₂AlCl, RSnO₃SCF₃ and R₃B, where R is an alkyl or aryl group, as described, for example, in U.S. Pat. No. 3,496,217, U.S. Pat. No. 3,496,218 and U.S. Pat. No. 4,774,353. According to U.S. Pat. No. 3,773,809, the promoter used may be a metal in cationic form which is selected from the group consisting of zinc, cadmium, beryllium, aluminum, gallium, indium, thallium, titanium, zirconium, hafnium, erbium, germanium, tin, vanadium, niobium, scandium, chromium, molybdenum, tungsten, manganese, rhenium, palladium, thorium, iron and cobalt, preferably zinc, cadmium, titanium, tin, chromium, iron and cobalt, and the anionic moiety of the compound may be selected from the group consisting of halides such as fluoride, chloride, bromide and iodide, anions of lower fatty acids having from 2 to 7 carbon atoms, HPO₃²⁻, H₃PO²⁻, CF₃COO⁻, C₇H₁₅OSO₂⁻ or SO₄²⁻. Further suitable promoters disclosed by U.S. Pat. No. 3,773,809 are borohydrides, organoborohydrides and boric esters of the formula R₃B and B(OR)₃, where R is selected from the group consisting of hydrogen, aryl radicals having from 6 to 18 carbon atoms, aryl radicals substituted by alkyl groups having from 1 to 7 carbon atoms and aryl radicals substituted by cyano-substituted alkyl groups having from 1 to 7 carbon atoms, advantageously triphenylboron. Moreover, as described in U.S. Pat. No. 4,874,884, it is possible to use synergistically active combinations of Lewis acids, in order to increase the activity of the catalyst system. Suitable promoters may, for example, be selected from the group consisting of CdCl₂, FeCl₂, ZnCl₂, B(C₆H₅)₃ and (C₆H₅)₃SnX, where X═CF₃SO₃, CH₃C₆H₄SO₃ or (C₆H₅)₃BCN, and the preferred ratio specified of promoter to nickel is from about 1:16 to about 50:1.

In the context of the present invention, the term Lewis acid also includes the promoters specified in U.S. Pat. No. 3,496,217, U.S. Pat. No. 3,496,218, U.S. Pat. No. 4,774,353, U.S. Pat. No. 4,874,884, U.S. Pat. No. 6,127,567, U.S. Pat. No. 6,171,996 and U.S. Pat. No. 6,380,421.

Particularly preferred Lewis acids among those mentioned are in particular metal salts, more preferably metal halides, such as fluorides, chlorides, bromides, iodides, in particular chlorides, of which particular preference is given to zinc chloride, iron(II) chloride and iron(III) chloride.

According to the invention, the reaction is carried out in the presence of a hydrocarbon which leads under certain pressure, concentration and temperature conditions to the formation of at least two liquid phases of the overall system, of which one phase has a higher proportion of the Ni(0)-containing catalyst, based on the total weight of this phase, than the other phase or other phases.

In the context of the present invention, a hydrocarbon is a single hydrocarbon or a mixture of hydrocarbons.

Hydrocarbon should advantageously have a boiling point of at least 30° C., preferably at least 60° C., in particular at least 90° C., at a pressure of 10⁵ Pa.

Hydrocarbon should advantageously have a boiling point of at most 140° C., preferably at most 135° C., in particular at most 130° C., at a pressure of 10⁵ Pa.

Suitable hydrocarbons are described, for example, in U.S. Pat. No. 3,773,809, column 3, lines 50-62.

Preference is given to a hydrocarbon selected from the group consisting of cyclohexane, methylcyclohexane, cycloheptane, n-hexane, n-heptane, n-octane, isooctane and mixtures thereof, in particular from methylcyclohexane, n-heptane, isomeric heptanes, n-octane, isomeric octanes such as 2,2,4-trimethylpentane and mixtures thereof, more preferably methylcyclohexane, n-heptane, 2,2,4-trimethylpentane, n-octane, octane isomer mixture and mixtures thereof.

With particular preference, a hydrocarbon, in the context of this invention-meaning a single hydrocarbon or else a mixture of such hydrocarbons, can be used for removal, in particular by extraction, of adiponitrile from a mixture comprising adiponitrile and an Ni(0)-containing catalyst, said hydrocarbon having a boiling point in the range between 90° C. and 140° C. From the mixture obtained after the removal according to this process, the adiponitrile may advantageously be obtained by distillative removal of the hydrocarbon, and the use of a hydrocarbon having a boiling point within the range specified allows a particularly economical and technically simple removal by the possibility of condensing the distilled-off hydrocarbon with river water.

The hydrocyanation can be carried out in a manner known per se, for example in accordance with the documents specified in this description.

In general, such hydrocyanations can be carried out at a temperature in the range from −50° C. to 200° C. and a pressure in the range from 0.05 to 100 bar, while a temperature in the range from −15° C. to 75° C. and a pressure in the range from 0.05 to 10 bar have been found to be advantageous.

It has been found that, surprisingly, when carrying out the process according to the invention, the presence of the hydrocarbon defined in accordance with the invention results in no impairment of the hydrocyanation compared to such a hydrocyanation in the absence of such a hydrocarbon, for example a reduction in the catalyst activity to be expected as a result of dilution.

In a preferred embodiment, after the hydrocyanation, the overall system may be placed under pressure, concentration and temperature conditions which lead to the formation of at least two liquid phases, of which one phase has a higher proportion of the Ni(0)-containing catalyst, based on the total weight of this phase, than the other phase or other phases, and then

said phase which has a higher proportion of the Ni(0)-containing catalyst, based on the total weight of this phase, than the other phase or other phases may be removed from the overall system.

For phase separation, a wide pressure, concentration and temperature range can generally be selected, and the optimum parameters of the particular composition of the reaction mixture can be determined easily by a few simple preliminary experiments.

An advantageous temperature has been found to be at least 0° C., preferably at least 20° C.

An advantageous temperature has been found to be at most 100° C., preferably at most 60° C.

An advantageous pressure has been found to be at least 0.1 bar, preferably at most 0.5 bar.

An advantageous pressure has been found to be at most 10 bar, preferably at most 5 bar.

Phase separation can be carried out in one or more apparatus known per se for such phase separation.

In an advantageous embodiment, the phase separation can be carried out in the same reactor in which the hydrocyanation of the process according to the invention is likewise carried out, for example by equipping this reactor with a calming zone.

The phase separation results in two liquid phases, of which one phase has a higher proportion of the Ni(0)-containing catalyst, based on the total weight of this phase, than the other phase or other phases.

Advantageously, the phases are separated from one another, in particular said phase which has a higher proportion of the Ni(0)-containing catalyst, based on the total weight of this phase, than the other phase or other phases, is removed from the overall system.

Said phase contains the predominant portion of Ni(0) and of the phosphorus compound suitable as a ligand for Ni(0). When the ligand used is a mixture of at least one monodentate ligand and of at least one bidentate ligand, there is generally accumulation of the bidentate ligand in said phase relative to the monodentate ligand compared to the one or more further phases. This is particularly advantageous since, in this advantageous embodiment, the bidentate ligands, which are typically more thermally sensitive than the monodentate ligands, are converted to an easily recyclable form, while the monodentate ligands, which can be thermally stressed, may optionally be removed from the one or more further phases by separating processes involving little thermal stress, such as extraction, or else by processes involving thermal stress, such as distillation.

Organic mono- and dinitriles, Lewis acid and any catalyst decomposition products formed are substantially present in one or more of the one or more other phases.

In a preferred embodiment, said phase containing the predominant portion of Ni(0) and of the phosphorus compound suitable as a ligand for Ni(0) is recycled into a hydrocyanation of an olefinically unsaturated compound of the process according to the invention.

1. A process for hydrocyanating an olefinically unsaturated nitrile in the presence of an Ni(0)-containing catalyst, which comprises carrying out the reaction in the presence of a hydrocarbon which leads under certain pressure, concentration and temperature conditions to the formation of at least two liquid phases of the overall system, of which one phase has a higher proportion of the Ni(0)-containing catalyst, based on the total weight of this phase, than the other phase or other phases.

2. A process as claimed in claim 1, wherein the proportion of the hydrocarbon in the entire reaction mixture is in the range from 10 to 50% by weight.

3. A process as claimed in claim 1, wherein the hydrocarbon has a boiling point in the range from 30 to 140° C. at a pressure of 10⁵ Pa.

4. A process as claimed in claim 1, wherein the hydrocarbon is selected from the group consisting of cyclohexane, methylcyclohexane, cycloheptane, n-hexane, n-heptane, n-octane, isooctane and mixtures thereof.

5. A process as claimed in claim 1, wherein the catalyst, in addition to Ni(0), additionally contains a compound which is suitable as a ligand for Ni(0) and has at least one trivalent phosphorus atom, or a mixture of such compounds.

6. A process as claimed in claim 5, wherein the compound suitable as a ligand is selected from the group consisting of monophosphinite, monophosphonite, monophosphite and mixtures thereof.

7. A process as claimed in claim 5, wherein the compound suitable as a ligand is selected from the group consisting of diphosphinite, diphosphonite, diphosphite, phosphinite-phosphonite, phosphinite-phosphite, phosphonite-phosphite and mixtures thereof.

8. A process as claimed in claim 5, wherein the compound suitable as a ligand which is used is a mixture of two components
whose first component is selected from the group consisting of monophosphinite, monophosphonite, monophosphite and mixtures thereof and
whose second component is selected from the group consisting of diphosphinite, diphosphonite, diphosphite, phosphinite-phosphonite, phosphinite-phosphite, phosphonite-phosphite and mixtures thereof.

9. A process as claimed in claim 1, wherein the olefinically unsaturated compound to be hydrocyanated which is used is a pentenenitrile or a mixture of isomeric pentenenitriles.

10. A process as claimed in claim 1, wherein, after the hydrocyanation, the overall system is placed under pressure, concentration and temperature conditions which lead to the formation of at least two liquid phases, of which one phase has a higher proportion of the hydrocarbon, based on the total weight of this phase, than the other phase or other phases, and then
said phase which has a higher proportion of the hydrocarbon, based on the total weight of this phase, than the other phase or other phases is removed from the overall system.

11. A process as claimed in claim 10, wherein the removed phase is recycled into the hydrocyanation of an olefinically unsaturated compound.

12. The use of a hydrocarbon having a boiling point in the range between 90 and 140° C. for removing adiponitrile from a mixture comprising adiponitrile and an Ni(0)-containing catalyst.

13. A process for removing adiponitrile from a mixture comprising adiponitrile and an Ni(0)-containing catalyst using a hydrocarbon which has a boiling point in the range between 90 and 140° C.

14. A process as claimed in claim 2, wherein the hydrocarbon has a boiling point in the range from 30 to 140° C. at a pressure of 10⁵ Pa.

15. A process as claimed in claim 2, wherein the hydrocarbon is selected from the group consisting of cyclohexane, methylcyclohexane, cycloheptane, n-hexane, n-heptane, n-octane, isooctane and mixtures thereof.

16. A process as claimed in claim 3, wherein the hydrocarbon is selected from the group consisting of cyclohexane, methylcyclohexane, cycloheptane, n-hexane, n-heptane, n-octane, isooctane and mixtures thereof.

17. A process as claimed in claim 2, wherein the catalyst, in addition to Ni(0), additionally contains a compound which is suitable as a ligand for Ni(0) and has at least one trivalent phosphorus atom, or a mixture of such compounds.

18. A process as claimed in claim 3, wherein the catalyst, in addition to Ni(0), additionally contains a compound which is suitable as a ligand for Ni(0) and has at least one trivalent phosphorus atom, or a mixture of such compounds.

19. A process as claimed in claim 4, wherein the catalyst, in addition to Ni(0), additionally contains a compound which is suitable as a ligand for Ni(0) and has at least one trivalent phosphorus atom, or a mixture of such compounds.

20. A process as claimed in claim 2 wherein the olefinically unsaturated compound to be hydrocyanated which is used is a pentenenitrile or a mixture of isomeric pentenenitriles.
